(19) United States
(12) Patent Application Publication
(10) Pub. No.: US 2023/0248385 A1
(43) Pub. Date: Aug. 10, 2023

(54) WOUND DEBRIDEMENT CLOTH

(71) Applicant: LOHMANN & RAUSCHER GMBH, SCHONAU/TRIESTING (AT)

(72) Inventors: CARINA TAUSCHER, Markt Piesting (AT); CORNELIA HAUSLER, Vienna (AT)

(21) Appl. No.: 18/009,058

(22) PCT Filed: Jun. 10, 2021
(86) PCT No.: PCT/EP2021/065571

§ 371 Date: Feb. 2, 2023

(30) Foreign Application Priority Data
Jun. 10, 2020 (DE) 20 2020 103 353.0
Jul. 3, 2020 (DE) 10 2020 117 622.8

(51) Int. Cl. A61B 17/32; A61F 13/00; A61F 13/15; A61F 15/00

(52) U.S. Cl. CPC A61B 17/32; A61F 13/00991; A61F 13/15577; A61F 15/001; A61B 2017/320004; A61B 2017/00761

(57) ABSTRACT

Wound cleaning device having a carrier layer and a wound cleaning layer arranged thereon, designed for holding wound exudate and/or solid wound components and having cleaning threads, the carrier layer and/or the wound cleaning layer being provided with at least one storage material that increases the capacity for holding wound exudate.

The invention relates to a wound cleaning cloth having a carrier layer and arranged thereon, a wound cleaning layer designed for absorbing wound exudate and/or solid wound material, such as fibrin plaques, dead subcutaneous tissue, such as excess horn material or dead horn cells and/or necroses, and having cleaning threads for detaching the wound components from the wound.

Such wound cleaning devices are, for example, described, in WO 2010/085831 A1 and PCT/EP/2019/073568. The disclosure content of these documents with regard to the properties of the carrier layer and the wound cleaning layer as well as the threads is hereby incorporated in this description by express reference.

Wound cleaning cloths of the type described above are used, for example, for the so called debridement. This involves the wound bed preparation process, during which substances or human material formed by the body itself, such as excess fluid (wound exudate), fibrin plaques, dead epidermal tissue, such as excess horn material or dead horn cells and/or plaques of dead tissue (necroses), are removed.

Using the prior art wound cleaning devices, the substances to be removed can be detached from the wound by means of the threads and held between the threads. When using the prior art wound cleaning devices, it has, however, been shown that during the wound cleaning, the substances to be removed become detached again from the wound cleaning layer and get back into the wound.

In view of these problems in the state of the art, the invention is based on the objective of providing wound cleaning devices that have an improved cleaning effect.

According to the invention, this objective is achieved by an enhancement to the prior art wound cleaning devices, which is essentially characterized in that the carrier layer and/or the cleaning layer is provided with at least one storage material that increases the capacity for holding wound exudate.

The invention is based on the finding that the deficiencies observed when using the prior art wound cleaning devices are primarily due to the fact that the holding capacity of the wound cleaning device during the wound cleaning process is attained, and subsequently, removed substances get back into the wound. This deficiency is corrected according to the invention in that suitable storage materials are used. In doing so, surprisingly, it has been shown that, despite the volume occupied by the storage materials, overall, more storage capacity can be attained because, on the one handy, the available storage space of the wound cleaning device can be better utilized and/or, on the other hand, fully utilizing the storage materials, the capacity overall is optimized by proper integration of the wound exudate in the wound cleaning layer and/or carrier layer. This also prevents wound exudate from being smeared around on the surrounding skin and reduces the infection risk thereby caused.

Within the scope of the invention, the holding capacity of the wound cleaning device for essentially liquid substances is described by the so called “fluid holding capacity”. For determining this “fluid holding capacity”, the wound cleaning device is first weighed. Thereafter, the wound cleaning device is placed into a wound exudate solution for a period of 60 seconds. Subsequently, the wound exudate solution-saturated product is grasped using a forceps and held in a vertical direction for a period of 30 seconds to allow solution to drip off the wound cleaning device. Thereafter, the product is horizontally placed into a dish and weighed again. Based on the weighing results, it is determined bow many milliliters of the exudate solution per gram of the wound cleaning device are retained in the wound cleaning device. This value is the wound cleaning capacity in accordance with the invention herein. Should the value be increased by adding storage materials, within the scope of the invention herein, it means an increase in the wound exudate holding capacity.

Similar to conventional wound cleaning devices, the wound cleaning threads of wound cleaning devices according to the invention may include plastic fibers, in particular polyester fibers and/or polypropylene fibers, in particular consisting of polyester fibers and/or polypropylene fibers. Hence, in this arrangement, the cleaning threads may be embodied as monofilaments, ergo one fiber per thread, and/or as multifilaments, ergo, as the case may be, as twisted fiber bundles. Within the scope of the invention, the cleaning threads may form loops, in which the threads emerge from the carrier layer and reenter into the carrier layer after loop formation. Alternatively or additionally, the threads may have freely cantilevered ends, i.e. they emerge from the carrier layer and have free ends.

Within the scope of the invention, it has proven to be particularly advantageous if the proportion of storage materials, based on the dry weight of carrier layer and cleaning layer (total weight), is 0.1%-25% by weight, in particular 0.15%-20% by weight, preferably 0.2%-17% by weight. If the proportion of the storage materials is increased beyond the stated values, this may lead to adhesion of the cleaning threads to each other which, in turn, may entail a reduction of the storage capacity. In case of a reduction in the proportion of storage materials below the stated values, the effect of the storage materials becomes barely noticeable.

It has been shown that even within the carrier layer, the storage materials can develop an effect that increases the holding capacity. It has, therefore, been proven to be expedient to base the amount of the storage materials on the total weight of the wound cleaning device. Care must, however, also be taken to ensure that the properties of the wound cleaning threads are not excessively impaired by the storage materials and that the available holding capacity between the cleaning threads is not excessively reduced by the storage materials. Considering these aspects, it has proven to be advantageous if the proportion of the storage material, based on the dry weight of the wound cleaning layer, is 0.2%-30% by weight, in particular 0.25%-27% by weight, preferably 0.3%-25% by weight.

Within the scope of the invention, it has proven to be particularly advantageous if at least the wound cleaning layer is impregnated using a preferably aqueous solution of the storage material. In this way, adhesion of the cleaning threads by dried storage materials can be prevented.

Within the scope of the intended increase in the storage capacity, it has proven to be expedient if the concentration of the storage material in the solution used to impregnate the cleaning layer is 0.1% or greater, preferably 0.3% or greater, and/or 5% or less, particularly 2.5% or less, particularly preferred 1.5% or less. An increase in the concentration requires an increase in the viscosity of the solution. This may impair the homogeneous distribution of the storage materials in the cleaning layer. If the concentration is too low, the available cleaning layer capacity is mostly already attained by the impregnating solution, so that only a small residual capacity remains available for the wound exudate and the other substances to be removed from the wound. For this purpose, the concentration is given as the weight of the storage material based on the weight of the solvent. Hence, when using water as the solvent, a concentration of 0.1% means 1 g of storage material per liter of solvent.

Within the scope of the invention, it has proven to be particularly expedient if the viscosity of the solution is adjusted to 2,000 mPa·s or less, in particular 1,000 mPa·s or less, measured according to DIN ISO 2555, using a Model RTV Brookfield Viscosimeter.

The storage material used within the scope of the invention may have at least one surfactant and/or at least one substance that swells on contact with water or an aqueous solution. Within the scope of the invention, a surfactant will be understood to be a hydrophilizing substance, which enables hydrophobic substances to dissolve. Surfactants or emulsifiers per se lower the energy of the phase limit (surface or interfacial tension). This counteracts segregation. The properties of surfactants or emulsifiers of blending and stabilizing an immiscible liquid to form a finely distributed mixture promotes the desired increase in the holding capacity for wound exudate within the scope of wound cleaning. The reduction of the surface tension using a surfactant also facilitates the penetration of the wound exudates into the spaces between the wound cleaning threads and, as a result, increases the holding capacity overall.

With a substance that swells on contact with water or an aqueous solution, the water is bound in the form of a gel, This leads to an increase in the holding capacity overall, because the already bound water or wound exudate cannot drip back out of the cleaning layer.

A storage material that can be used within the scope of the invention may contain at least one cellulose ether, in particular hydroxyethyl cellulose; carboxymethyl cellulose; methyl cellulose; ethyl cellulose and/or hydroxypropyl cellulose; other polysaccharides, such as starch, dextrans, chitosan, carrageenan, and alginates; milk protein; hyaluronic acid: sugar alcohols; glycols; salts and/or mixtures thereof.

Wound healing can be further enhanced if the wound cleaning layer and/or the carrier layer is blended using anti inflammatory and/or antimicrobial substances, such as WO₃ (tungsten(VI) oxide). In this case, the antibacterial effect of WO₃ is attributed to the absorption of water by the tungsten oxide, thereby forming a thin H⁺ ion-enriched layer, which may damage bacteria membranes. It is, of course, possible to use other antimicrobial and/or anti inflammatory substances additionally or alternatively, too. In doing so, within the scope of the invention, use can also be made of the anti inflammatory/antimicrobial effect of the storage materials per se, such as hydroxyethyl cellulose.

Within the scope of the invention, it is contemplated to spray solutions, in particular aqueous solutions of the storage materials, onto the boundary surface of the wound cleaning layer that faces away from the carrier layer, potentially using a spray bottle nozzle, In alternative methods, a solution of the storage material is introduced into the pad by pipetting at regular intervals. In this process, moistening of the cleaning layer takes place starting at the fiber base facing the carrier layer. Within the scope of the invention, it has, however, proven to be particularly expedient to introduce the storage materials by immersing the wound cleaning device in an appropriate solution. For this purpose, the following procedure is used:

In one embodiment of the invention, cellulose is used as storage material. Cellulose is the polysaccharide most frequently available in nature and has many beneficial properties. They include biodegradability, biocompatibility, absence of toxicity, low cost as well as good thermal and chemical stability. The combination of the properties of high biocompatibility with the absence of toxicity and nonexistence of immunogenic activity has proven to be particularly advantageous within the scope of the invention.

The storage material, such as hydroxyethyl cellulose (HEC), is added to distilled water until the desired concentration of the storage material in the solution is obtained and is continuously agitated for one hour at elevated temperatures of, for example, approx. 60° C., in order to obtain a homogeneous mixture. In exemplary embodiments of the invention, solutions at concentrations of 0.3% (3 g/L) and 2.5% (25 g/L) HEC are produced. When using hydroxyethyl cellulose as a storage material, 0.5% citric acid is added as a crosslinking agent. When using citric acid as a crosslinking agent, a spongy, porous material can be obtained. This property is attributed to the strong hydrogen bonding between the polymer chains. As a result of these strong interactions, a very large capacity for absorbing large amounts of water can be obtained. The structure thus obtained can substantially swell without being dissolved in the aqueous solution. The resulting solution is mixed for 2 hours at elevated temperatures of, for example, approx. 60° C., in order to achieve crosslinking, and is poured into a glass dish without any prior cooling. Thereafter, the wound cleaning device is placed into the solution with the boundary surface of the wound cleaning layer facing away from the carrier layer facing downwards, so that the solution can be uniformly accommodated in the cleaning layer and/or the carrier layer, potentially taking advantage of the capillary effect between the cleaning threads.

In embodiments of the invention, polyester monofilaments having a thread size of about 1 dtex (1 g/10,000 m) and a pile height (see below) of 5 mm can be provided on the boundary surface of the wound cleaning device facing away from the carrier layer. The mass of the cleaning threads (monofilaments) may be 5 g/100 cm² of area.

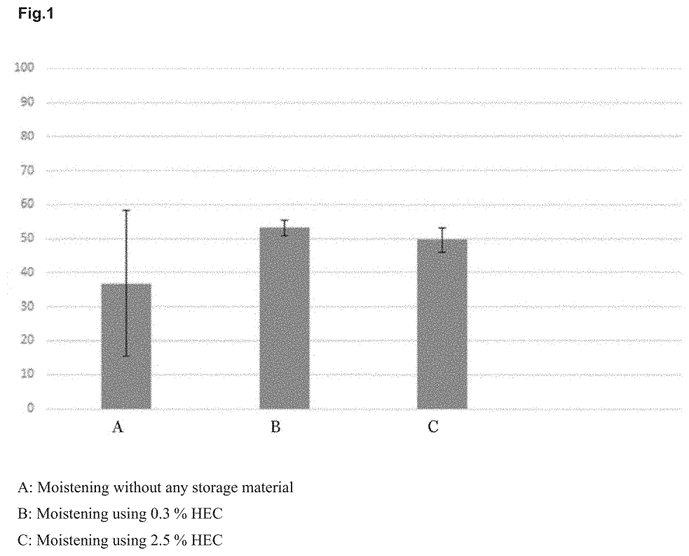

FIG. 1 shows the “fluid holding capacity” of the wound cleaning devices obtained in this way in comparison to wound cleaning devices without any storage material merely premoistened using distilled water. The amount of exudate accommodated, based on the weight of the wound cleaning device, is given in percent.

Additionally or alternatively, the wound cleaning device can be blended with a storage material solution obtained without any special temperature treatment and/or without using a crosslinking agent or can be placed into an appropriate solution. Within the scope of the invention, it is contemplated to first dry the wound cleaning device obtained in this way before use and not to remoisten it until used. This can, however, cause the cleaning threads to adhere to each other, thus impairing the cleaning effect. For this reason, within the scope of the invention, it has proven to be particularly advantageous if the wound cleaning device is available in the form of a pack having a fluid impermeable sheath and a wound cleaning device, such as a wound cleaning cloth, held therein. Adhesion of the cleaning threads to each other during transport and storage can thus be avoided.

In an alternative embodiment of the invention, a kit for producing a wound cleaning device according to the invention or a wound cleaning cloth according to the invention is provided, having a dry wound cleaning device or a dry wound cleaning cloth and a preferably aqueous solution of at least one storage material. Before using the wound cleaning device, the wound cleaning layer can be impregnated using the solution provided in the kit, in order to obtain in this manner a design of the cleaning layer and/or the carrier layer with the storage material

In this arrangement, a kit according to the invention can be made available in the form of a so called “click pack”, In this click pack, wound cleaning device and storage material solution are held in separate chambers. By creasing the pack at a specified location, a connection between the chambers can be produced and thereby impregnation of the wound cleaning device using the storage material solution can be brought about.

Within the scope of the invention, the use of hydroxyethyl cellulose as a storage material has proven to be particularly expedient. This material is heat-resistant and is also suitable for sterilization processes using high temperatures. When using this storage material, it has proven to be particularly preferable to moisten the wound cleaning device using an aqueous solution of the storage material and subsequently to store it in a fluid impermeable sheath. Hydroxyethyl cellulose can be used in the form of an aqueous solution at a concentration range usually between 0.1% and 1.5%. Within the scope of the invention, it is particularly expedient to use hydroxyethyl cellulose solutions having concentrations between 0.1% and 5% for impregnating the wound cleaning device. At higher concentrations, the solution becomes very viscous and almost impossible to apply.

Within the scope of this invention, independent protection is claimed for the use of a storage material for a wound cleaning device for wound bed preparation, in which substances formed by the body itself, such as excess fluids, fibrin plaques, dead epidermal tissue, such as excess horn material and/or dead horn cells and/or dead tissue plaques (necroses) are removed. Within the scope of the utilization according to the invention, use can also be made of anti-microbiological, disinfecting, wound healing-promoting, film forming, moisturizing and/or an acidic environment-creating properties of the storage material in connection with the wound cleaning device.

When using hydroxyethyl cellulose as storage material, use can be made of the property of hydroxyethyl cellulose as a mucoadhesive polymer. This means that it bonds very well to mucous membranes. Because the surface condition of wounds and mucous membranes can be considered to be similar, hydroxyethyl cellulose adheres equally well to wound surfaces. As a result, appropriate film formation is promoted. The hydroxyethyl cellulose film prevents the wound from drying out and allows moisture to be retained in the wound, thereby promoting wound healing.

In a wound cleaning device according to the invention, the cleaning threads may exist in pile form. In this arrangement, the pile mass, i.e. the mass of the cleaning layer, may be 1-10 g/100 cm² of area. In this case, the cleaning layer may have monofilaments of polyester, which may have between 0.5 and 20 dtex. The threads may have threads projecting from the carrier layer, with freely cantilevered ends, while the shape of the threads may instead be curved. The length of the thread sections (pile height) available for wound cleaning outside the carrier layer may be 0.1 mm-50 mm, in particular 1 mm-40 mm, preferably 3 mm-30 mm, particularly preferred 4 mm-12 mm, such as approx. 5 mm or 10 mm. Neither do they need to project perpendicularly from the carrier layer. The carrier layer may feature a nonwoven, woven or knitted fabric. Alternatively or additionally, the carrier layer may instead be designed as a membrane or film, in which the cleaning threads are partially accommodated. With regard to the production of wound cleaning devices or cloths, which can be used within the scope of the invention, reference is made to WO 2010/085831 A1, which is included in this description by express reference in this regard, too.

In addition or as an alternative to hydroxyethyl cellulose, wound cleaning devices according to the invention may, among others, be provided with the following storage materials, where storage material solutions can be used that are produced in a manner similar to that described for hydroxyethyl cellulose and where the wound cleaning device can be provided with the storage material by placing it in appropriate storage material solutions, by spray-on and/or by using a pipette:

### Additional Surfactants

Poloxamers are block copolymers of ethylene oxide and propylene oxide. It is a debriding and antimicrobial surfactant. Poloxamer 188 attains increased heat stability (up to 260° C.) by propoxylation, which makes the substance compatible with common sterilization processes using heat. In addition to the increased heat stability, propoxylation can achieve preservation, anti oxidation, anti foaming and a viricidal effect. Optionally, butyl hydroxy toluene may be added. As additional surfactants, Tween 20/Tween-180, esterquats, sodium dodecyl sulfate, betaine, EHG (ethyl hexyl glycerol), carboxymethyl cellulose, methyl cellulose, ethyl cellulose, hydroxypropyl cellulose can be used within the scope of the invention.

### Milk Protein

For producing wound cleaning devices according to the invention, powdered milk (fat/protein/milk sugar) can, for example, be used. In this respect, milk protein also acts as an emulsifier.

### Acids

Acids have antimicrobial properties. At a value of 4.5 to 5.5, the natural pH of the skin is situated in the slightly acidic range. The reason thereof is that a slightly acidic environment helps the skin kill pathogens from the outside. Within the scope of the invention, acetic acid, lactic acid, citric acid; formic acid and the like can be used for producing wound cleaning devices according to the invention.

Hyaluronic acid is moisturizing; water bridges form (binding water), and the surface tension is reduced. Since the hyaluronic acid is significant for the exchange of information between the cells, it also has a positive effect in the wound healing process. It ensures that wounds heal without leaving any visible scars. This can be exploited within the scope of the invention herein.

### Sugar Alcohols

Sugar alcohols, such as glycerol, have water binding properties. The water binding effect can be used for increased accommodation of wound exudate. When using sugar alcohols, in particular glycerol, an antimicrobial effect was observed, too.

### Glycols

Within the scope of the invention, glycols, such as PEG 500/3000 (polyethylene glycol) can also be used for the production of wound cleaning devices according to the invention. PEG is water-soluble and highly hygroscopic. Within the scope of the invention, the ability to absorb water can be used to increase the capacity of wound cleaning devices. Additionally, PEG also has an antimicrobial effect.

### Salts

Within the scope of the invention, use can be made of the odor absorbing, disinfecting and water binding effect of salts. In the scope of the invention, salts serve as a storage material. They may additionally have antimicrobial properties and neutralize the odor of wounds. Within the scope of the invention, Ringer's solution, consisting of sodium chloride, potassium chloride and calcium chloride, lactates (lactic acid salts and esters), sodium acetate (sodium salt of acetic acid), table salt and salts of acids, can be used as storage material. Blends of the storage materials discussed above can also be used.

### Palmitoyl Oligopeptide

This storage material, a natural tripeptide, which is usable within the scope of the invention, has a biological effect in connection with the formation of new glycosaminoglycans and collagens and brings about increased hydration.

1. Wound cleaning device having a carrier layer and, arranged thereon, a wound cleaning layer designed to hold wound exudate and/or solid wound components and having cleaning threads, characterized in that the carrier layer and/or the wound cleaning layer is provided with at least one storage material that increases the holding capacity for wound exudate.

2. Wound cleaning device according to claim 1, characterized in that the cleaning threads have plastic fibers, in particular polyester fibers, in particular consist thereof.

3. Wound cleaning device according to claim 1 or claim 2, characterized in that the cleaning threads are designed as monofilaments and/or multifilaments.

4. Wound cleaning device according to any one of the preceding claims, characterized in that the cleaning threads form loops and/or have freely cantilevered ends.

5. Wound cleaning device according to any one of the preceding claims, characterized in that, based on the dry weight of carrier layer and cleaning layer, the proportion of at least one storage material is 0.1% 25% by weight, in particular 0.15%-20% by weight, preferably 0.2%-17% by weight.

6. Wound cleaning device according to any one of the preceding claims, characterized in that, based on the dry weight of cleaning layer, the proportion of at least one storage material is 0.2%-30% by weight, in particular 0.25%-27% by weight, preferably 0.3%-25% by weight.

7. Wound cleaning device according to one of the preceding claims, characterized in that at least the cleaning layer is impregnated using a preferably aqueous solution of the storage material.

8. Wound cleaning device according to claim 7, characterized in that the concentration of the storage material in the solution is 0.1% or more, preferably 0.3% or more and/or 5% or less, in particular 2.5% or less, particularly preferred 1% or less.

9. Wound cleaning device according to any one of the preceding claims, characterized in that the storage material has at least one surfactant and/or at least one substance that swells on contact with water or an aqueous solution.

10. Wound cleaning device according to any one of the preceding claims, characterized in that the storage material contains at least one cellulose ether, in particular hydroxyethyl cellulose, carboxymethyl cellulose, methyl cellulose, ethyl cellulose and/or hydroxypropyl cellulose, additional polysaccharides, such as dextrans, starch, chitosan, carrageenan and alginates, milk protein, hyaluronic acid, sugar alcohols, glycols, salts and/or blends thereof.

11. A process for producing a wound cleaning device according to any one of the preceding claims, in which at least the wound cleaning layer of the wound cleaning device is impregnated using a preferably aqueous solution of the storage material.

12. Pack having a fluid impermeable sheath and a wound cleaning device according to any one of the preceding claims held therein.

13. Kit for producing a wound cleaning device according to any one of the preceding claims, having a dry wound cleaning cloth and a preferably aqueous solution of at least one storage material.

14. Use of a storage material for a wound cleaning device for wound bed preparation, wherein substances formed by the body itself, such as excess fluids, fibrin plaques, dead tissue of the epidermis, such as excess horn material or dead horn cells and/or plaques from dead tissue (necroses), are removed.
